(19) United States
(12) Patent Application Publication
(10) Pub. No.: US 2024/0197204 A1
(43) Pub. Date: Jun. 20, 2024

(54) WEARABLE DEVICE

(71) Applicant: CHARCO NEUROTECH LTD, London (GB)

(72) Inventors: Soo Min JUNG, Cambridge (GB); Floyd PIERRES, Cambridge (GB)

(21) Appl. No.: 18/250,495

(22) PCT Filed: Oct. 1, 2021
(86) PCT No.: PCT/IB2021/059043

§ 371 Date: Apr. 25, 2023

(30) Foreign Application Priority Data
Oct. 26, 2020 (GB) 2016978.5

(51) Int. Cl. A61B 5/11; A61B 5/00; A61N 1/04; A61N 1/36

(52) U.S. Cl. CPC A61B 5/1101; A61B 5/0022; A61B 5/4836; A61B 5/683; A61N 1/0492; A61N 1/36067

(57) ABSTRACT

A wearable device is disclosed for managing tremors emanating from a body part of a user. The wearable device comprises a sensor that is configured to detect the tremor and transmit corresponding sensor data to at least one stimulating element, wherein the at least one stimulating element is configured to provide an electrical stimulus based on the sensor data. A dissipating portion of the wearable device is configured to increase an effective area for dissipating the electrical stimulus to the body. The dissipating portion is physically coupled with at least one stimulating element. The wearable device, when in operation, is in physical contact with body part of the user wherefrom the tremors emanate. The wearable device is stored in a docking station when not in operation. There is also disclosed a device integration application comprising software application to be executed by data processing arrangement of device.

### TECHNICAL FIELD

The present disclosure relates generally to wearable devices; more specifically, the present disclosure to medical wearable devices for managing tremors emanating from a body part of a user. Moreover, the present disclosure relates to a kit of parts and a device integration application.

### BACKGROUND

Currently, hundreds of millions of people worldwide have been diagnosed with various neurological disorders affecting their central and/or peripheral nervous systems. Demographic trends worldwide suggest that, approximately 10 million people suffer from Parkinson's disease while around 2.3 million people suffer from multiple sclerosis, amongst others. Generally, individuals who suffer from neurological disorders often experience difficulties related to muscles or movement, such as freeze of gait, stiffness, slowness, tremors or shaking. The impact of tremors, such as in Parkinson's disease, can be crucial in an individual's life, and affects an ability of such individuals to carry out normal daily activities. Moreover, in some cases, such tremors can lead to a level of discomfort and irritation, a loss of confidence and independence (such as a loss of financial independence due to loss of job) and depression. Over 50% of the Parkinson's patients are also reported to be suffering from depression.

Conventional approaches, for example for dealing with symptoms such as tremors, typically revolve around pharmacological products and surgical procedures (invasive or non-invasive), both of which are complex and costly. However, the pharmacological products may only temporarily help some people with tremor and are associated with several side effects such as sleepiness, poor concentration and coordination, muscle weakness, physical dependence and withdrawal symptoms. Moreover, the surgical procedures including deep brain stimulation surgery, thalamotomy and high-intensity focused ultrasound have been more effective as compared to pharmacological counterparts. However, the surgical procedures may lead to adverse conditions such as cognitive, psychiatric and/or behavioural changes and dysarthria (trouble speaking) and balance problems. Furthermore, even with optimal intervention, patients still require supplementary treatments (such as physical- and/or occupational therapy) to promote social involvement.

Recently, assistive and rehabilitative tremor-suppressing devices have been developed to control tremors while still allowing for voluntary movements. Such devices may for example be robotic exoskeletons typically based on impedance, biomechanical loading, self-balancing or counter-balancing techniques. However, such devices are extremely bulky and fail to control completely tremors due mainly to inefficient load transmission or actuating means. Moreover, such devices fail to monitor, manage and track the tremors, thereby not providing long-term support to the patients. Furthermore, the patients are dependent on their family or careers for recording their symptoms and daily activities and adhere to the prescribed medication schedules.

Moreover the techniques of nerve stimulation, such as Transcutaneous Electrical Nerve Stimulation (TENS) to treat tremors, stimulate the peripheral nerves that targets one or more individual nerves to reduce tremor. Such techniques pose great challenge owing to wide variation in wrist diameters, nerve locations, nerve depolarization characteristics, and skin conduction of the users.

Therefore, in light of the foregoing discussion, there exists a need to overcome the aforementioned drawbacks associated with conventional techniques for providing relief from tremors corresponding to neurological conditions, such as Parkinson's disease.

### SUMMARY

The present disclosure seeks to provide a wearable device for managing tremors emanating from a body part of a user. The present disclosure also seeks to provide a kit of parts comprising a wearable device and a docking station for receiving and storing the wearable device. The present disclosure further seeks to provide a device integration application comprising software application to be executed by a data processing arrangement of a device. The present disclosure seeks to provide a solution to the existing problem of controlling tremors currently managed by pharmacological and/or invasive surgical methods. An aim of the present disclosure is to provide a solution that overcomes at least partially the problems encountered in prior art, and to provide an efficient, non-pharmacological and non-invasive device for generating electrical stimulus based on a detection of an involuntary motor activity, i.e. a tremor. An aim of the present invention is to control tremors emanating from a muscle of a user, by muscle stimulation.

In one aspect, the present disclosure provides a wearable device for managing tremors emanating from a body part of a user, the wearable device comprising:
a sensor that is configured to detect a tremor and transmit corresponding sensor data to at least one stimulating element;
the at least one stimulating element that is configured to provide an electrical stimulus based on the sensor data; and
a dissipating portion that is configured to increase an effective area for dissipating the electrical stimulus to the body part, wherein the dissipating portion is physically coupled with the at least one stimulating element,
   wherein the wearable device, when in operation, is in physical contact with the body part of the user wherefrom the tremors emanate.

In another aspect, the present disclosure provides a kit of parts comprising:
a wearable device; and
a docking station for receiving and storing the wearable device, wherein the docking station is a single unit comprising a base and a lid removably attached at an end.

In yet another aspect, the present disclosure provides a device integration application comprising a software application to be executed by a data processing arrangement of a device, wherein the device is communicably coupled to a wearable device.

Embodiments of the present disclosure substantially eliminate or at least partially address the aforementioned problems in the prior art, and enable tremors associated with a neurological condition of a given individual to be controlled by electronic muscle stimulation produced by the wearable device as well as regulating stimulations applied to the individual that are based on a progress of the tremors.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1A and 1B are schematic illustrations of a wearable device, in accordance with various embodiments of the present disclosure;

FIG. 2 is a schematic illustration of a docking station, in accordance with an embodiment of the present disclosure;

FIG. 3 is a schematic illustration of a consumable adhesive pad, in accordance with an embodiment of the present disclosure; and

FIG. 4 is a schematic illustration of an exemplary implementation of a successful installation of the device integration application on a device, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, the present disclosure provides a wearable device for managing tremors emanating from a body part of a user, the wearable device comprising:
a sensor that is configured to detect a tremor and transmit corresponding sensor data to at least one stimulating element;
at least one stimulating element that is configured to provide an electrical stimulus based on the sensor data; and
a dissipating portion that is configured to increase an effective area for dissipating the electrical stimulus to the body part, wherein the dissipating portion is physically coupled with the at least one stimulating element,
   wherein the wearable device, when in operation, is in physical contact with the body part of the user wherefrom the tremors emanate.

In another aspect, the present disclosure provides a kit of parts comprising:
a wearable device; and
a docking station for receiving and storing the wearable device, wherein the docking station is a single unit comprising a base and a lid removably attached at an end.

In yet another aspect, the present disclosure provides a device integration application comprising a software application to be executed by a data processing arrangement of a device, wherein the device is communicably coupled to a wearable device.

The present disclosure provides a non-pharmacological, non-invasive, wearable device for controlling, relaxing or reducing tremors. The wearable device uses tremor-detecting sensors to detect a start and an end of a period of tremors, and to respectively generate or pause electronic muscle stimulation to control tremors emanating from a muscle of a user during the period. The wearable device is comfortable for the users, as it provides controlled vibrational stimulation with noise and heat dissipation. The wearable device is in physical contact with the body of the user, for example via a medical-grade adhesive for attachment to the user's skin. Beneficially, the wearable device provides improved social participation and a better quality of life for people with Parkinson's disease for example. Moreover, the wearable device is a standalone device requiring no complex arrangement, and may be configured based on the user's requirement using a simple device integrated application software, such as a mobile application, for recording and analysing the overall health (quality of life) of the user. Furthermore, the wearable device is tailored to suit the requirements of the user, such as in terms of altering variables of the stimulation (based on the severity of the tremors) to refine the pattern, strength and frequency from which the user experiences greatest benefits. Additionally, the wearable device provides improved social participation and a better quality of life for people with Parkinson's disease for example.

Throughout the present disclosure, the term “wearable device” as used herein refers to a system that is configured to be attachable to, detachable from and/or reattachable to a body part of the user, preferably a skin or muscle, to provide symptomatic relief from one or more symptoms associated with a disorder or disease condition. The wearable device has a proximal surface, and a distal surface opposite to the proximal surface. The proximal surface is configured to be attached to the body part of the user via suitable attachment means; for example, attachment is achieved via use of one or more straps. The term “wearable” as used herein refers to placement of the device at a suitable location rather than a slip-on arrangement. For example, the wearable device may be incorporated into one of a bracelet, a pendant, an anklet, an arm band, a wrist band or other item configured to be worn by a user such that the proximal surface thereof is arranged to contact the skin of the user, thereby maintaining a contact pressure, when the item is worn by the user. Beneficially, the wearable device may be configured to alleviate or eliminate physical symptoms of neurological conditions such as Parkinson's disease. Optionally, the wearable device of the present invention may be used by a person in addition to taking a prescribed medication, to help alleviate and manage any symptoms they are experiencing.

Throughout the present disclosure, the term “tremor” as used herein refers to an involuntary, rhythmic, oscillatory movement of one or more body parts. Such tremors are typically associated with muscle contraction and relaxation. Optionally, such tremors are a symptom of a disorder that affects a central or peripheral nervous system of a given person affected by the tremors. Optionally, the tremors are associated with at least one condition selected from: Parkinson's disease, Alzheimer's disease, essential tremor, multiple sclerosis, neural infection, neural death or damage, a shock (such as a spinal cord shock), a stroke or neurodegenerative disease, brain injury, chronic kidney disease, and so forth. Alternatively, tremors are associated with age, overuse and withdrawal of drugs or alcohol, mercury poisoning, hormonal imbalance (hypoglycaemia, hyperthyroidism), a lack of sleep, a lack of vitamins and other nutrients (such as thiamine and magnesium), increased stress, heightened strong emotions (anxiety, fear), fever, physical exhaustion, and so forth. More optionally, the body part is a tensed muscle from where the tremors originate. Optionally, the tremors may affect one or more body parts. Optionally, the body part is a skin (or muscle) of: a head, a sternum, an arm, a shoulder, a wrist, a hand, a neck, a trunk, an ankle, a leg, a foot, a temple, a face, an elbow, fingers, a spine, a knee, and so forth. Beneficially, the aforementioned body parts allow for an easy access of the wearable device by the user and/or by a carer or family member of an incapacitated user. However, some users may not be comfortable in showing off the wearable device and may wear the device at any other body part hidden under an item of cloth for example. For example, the wearable device may be placed against thighs or on a back region of the user.

The wearable device comprises a sensor that is configured to detect the tremor and transmit corresponding tremor-representative sensor data to at least one stimulating element. The sensor typically detects a movement, such as a fast movement or an abnormal movement. Optionally, the sensor is configured to detect a movement, such as a tremor, a shaking, and so on, consequent to a tensed muscle. Optionally, the sensor is selected from at least one of: a gyroscopic sensor, an accelerometer sensor, a magnetometer sensor, an electromyography (EMG) sensor, a flexural sensor, a stretch sensor, or a combination thereof. Optionally, the sensor data is obtained while the user is in motion or in a resting position. In this regard, the sensor records only the tremors and not the movements or displacement of the user. Optionally, the sensor data is at least one of: a start of tremor, an end of tremor, a severity of tremor, a frequency of tremor, a locus of tremor, an amplitude of tremor, a speed of tremor. Optionally, a start time and end time of tremors produce a change in the muscle of origin and are sensed and recorded by the sensor of the wearable device arranged to be positioned on the corresponding muscle.

The wearable device comprises at least one stimulating element that is configured to provide an electrical stimulus based on the sensor data. Optionally, the at least one stimulating element is an arrangement that is configured to transmit electrical stimulus or pulses to manage tremors. Moreover, the at least one stimulating element is triggered by a start of a tremor and arrested by an end of the tremor. The electrical stimulus or pulses typically mimic an action of signals induced by neurons, thereby targeting muscles or nerves therein. The at least one stimulating agent delivers a low-voltage electrical stimulus (electric current) to the targeted muscle via electrodes in order to result in muscle contraction and relaxation.

Optionally, the wearable device is configured to regulate, based on the sensor data, the at least one stimulating element to control one or more parameters of the electrical stimulus. The regulation of the at least one stimulating element is required to alleviate the tremors emanating from the targeted body part. It will be appreciated that the regulation of the at least one stimulating element is based on the sensor data associated with the tremor. Typically, the one or more parameters of the electrical stimulus is controlled based on a severity, a frequency, an intensity, an amplitude or a speed of the tremors sensed by the sensor. Notably, the one or more parameters of the electrical stimulus includes, but is not limited to, an intensity of the electrical stimulus, an amplitude of the electrical stimulus, a frequency of the electrical stimulus, a duration of the electrical stimulus, a type of the electrical stimulus. In an example, the electrical stimulus has a periodic output, such as a pulse-like stimulus or a continuous stimulus. Optionally, the pulse-like stimulus comprises a regular, rhythmic or periodic output (or fluctuations) that provide cue signs to modulate sensory dysfunction, while the continuous stimulus may be of high or low intensities resulting in a reduction or a relaxation of the sensory dysfunction.

The wearable device comprises a dissipating portion configured to increase an effective area for dissipating the electrical stimulus, wherein the dissipating portion is coupled with the at least one stimulating element. The term “dissipating portion” as used herein refers to a surface for transferring electrical stimulus from its site or origin, i.e. the at least one stimulating element, to another surface, such as a body part of the user. Through the dissipating portion, the electrical stimulus is provided to the target area covered by the dissipating portion, i.e. larger than the area of the at least one stimulating element. Beneficially, increasing an effective area of the dissipation of the electrical stimulus provided by the at least one stimulating element enables the effect of the electrical stimulus to be increased, for example maximised. The application of the electrical stimulus may therefore not be limited to specific local application reliant on direct contact with the at least one stimulating element. Rather, a surface area across which the electrical stimulus is to be applied may be increased significantly by providing a dissipating portion or desired size or specifications. This may reduce power requirements and/or reduce a number of stimulating elements required to provide the electrical stimulus across a larger surface area of the user. Optionally, the dissipating portion is fabricated from a material selected from a group of silicone, rubber, flexible plastic, foam, metal, alloys of metal, or any combination thereof. Optionally, the material is a flexible, elastic or viscoelastic material. Beneficially, the layer of flexible or elastic material, optionally silicone, reduces a noise (often disturbing) attributed to the one or more components of the wearable device, and provides electrical stimulus that is comfortable to the user.

Optionally, the dissipating portion is configured to receive (for example in a recess or aperture therein) or at least partially surround or encase the at least one stimulating element. More optionally, the dissipating portion is substantially hollow shaped, and comprises a closed end and an open end to form a recess extending into the hollow shape from the open end to receive the at least one stimulating element. Alternatively, the dissipating portion may comprise two open ends to form an aperture extending between the ends of the dissipating portion. Beneficially, the dissipating portion encloses and protects the at least one stimulating element from a potential damage during handling or using the wearable device. Optionally, a diameter or width of the dissipating portion is between substantially 10 mm and 40 mm. Optionally, the dissipating portion has a thickness of at least 3 mm. Optionally, the dissipating portion has a thickness of at least 3 mm in all dimensions of the dissipating portion for example in the absence of the recess or aperture. For example, in all directions surrounding the at least one stimulating element, the dissipating portion has a thickness in a range of 3 mm to 10 mm.

Optionally, the recess is configured to be substantially filled by a combination of the at least one stimulating element and one or more other components of the wearable device (for example, a power source such as a battery that is configured to power the at least one stimulating element). Optionally, the recess has a substantially similar shape to a shape of the dissipating portion or another shape (for example, a shape substantially similar to a shape of the at least one stimulating element and/or a shape of one or more other components of the wearable device).

Optionally, the dissipating portion comprises a substantially cylindrical shape having a proximal end placeable on the body of the user, and a distal end opposite to the proximal end. Optionally, the dissipating portion may have another shape, for example, a triangular, square, pentagonal or other polygonal prismatic shape. The proximal end of the dissipating portion forms at least a part of the proximal surface of the wearable device that is configured to contact (either directly or indirectly) the skin of the user for delivering the electrical stimulus thereto. Optionally, the at least on stimulating element forms a part of the surface that is configured to deliver the electrical stimulus to the user. It will be appreciated that a difference in the surface configured to deliver the electrical stimulus may enable different applications of electrical stimulus of the same magnitude to be experienced by the user. In other words, the part of the surface formed by the at least one stimulating element may be configured to deliver or transmit an electrical stimulus to the user at a different intensity (i.e. more-intense local electrical stimulus) than the part of the surface formed by the dissipating portion (i.e. less-intense local electrical stimulus). Beneficially, a combination of a more-intense local electrical stimulus and a less-intense broad electrical stimulus further improves the effect of the application of electrical stimulus on tremors and other symptoms such as freeze of gait, stiffness and slowness. Alternatively, the at least one stimulating element may not form (for example, may be set back from) a part of the surface.

Optionally, a capping layer is provided at the distal end of the dissipating portion to ensure the at least one stimulating element and any other components of the wearable device remain secured or located within the recess or aperture. Optionally, the at least one stimulating element and one or more other components of the wearable device may be secured within the recess or aperture using, for example, a friction fit (for contacting each other sufficiently closely, and with enough force, to prevent inadvertent escape thereof), or any suitable means known to a person skilled in the art. The capping layer is configured to form an outer surface of the wearable device. Optionally, the capping layer is fabricated from the same material as the dissipating portion.

Optionally, the wearable device comprises a housing. The housing may be configured to at least partially surround the dissipating portion. The housing may be fabricated from a plastics material (for example, polypropylene or polycarbonate) or a metal or alloy of metal (for example, aluminium). The housing may be configured to leave the surface configured to deliver the electrical stimulus to the user exposed. The housing may be configured to surround an axially extending portion of the dissipating portion (for example, the housing may not be configured to cover end faces, i.e. the proximal and distal ends, of the dissipating portion). The housing may protect the dissipating portion from impact, scratches or other degradation which could affect performance of the dissipating portion.

The wearable device, when in operation, is in physical contact with the body part of the user wherefrom the tremors emanate. In order to manage the tremors emanating from the body part, i.e. skin or muscle, by dissipating electrical stimulus provided by the at least one stimulating element thereto, the proximal end of the wearable device is arranged to physically contact the tremor-experiencing body part. Optionally, the physical contact with the body part of the user is by a mechanical engagement means, and wherein the mechanical engagement means is at least one of: an adhesive, a strap, a locket, a bracelet, a band, a belt, a vacuum cup, a magnet, a hook and loop fastener. The wearable device may be arranged to be held by a strap, a locket, a band or a belt, and worn to physically contact the body part as an accessory. Alternatively, the user may use adhesives, vacuum cups, magnet or hook and loop fastener as means of mechanical engagement of the wearable device with the body of the user to be free of additional elements such as those mentioned above.

It will be appreciated that any combination of the aforementioned mechanical engagement means may be used to arrange the wearable device in physical contact with the body part of the user. In an example, the mechanical engagement means is implemented as a hook and loop fastener. In such a case, one or more layers of adhesive is provided to affix the hook and loop fastener to the wearable device and the skin of the user via the hook side and the loop side thereof respectively. In such a case, the one or more layers of adhesive may be of different grades, such as a medical-grade adhesive or a strong adhesive.

Optionally, the layer of adhesive is configured to adhere the proximal surface of the wearable device to the skin of the user is a medical grade adhesive, for example, DuploMED® ELE77301. The adhesive permits easy positioning of the wearable device on the body part of the user for maintaining an effective contact pressure for the device to function. Optionally, the adhesive is configured to bond the surface to the skin of the user for an extended period of time, for example two or more days, such as three, four, five, six, seven, ten, most beneficially 14 or more days. The durability of the adhesive improves ease of operation for the user, by reducing fixation and removal of the wearable device from the skin of the user with the adhesive remaining on the skin for the extended period of time, while the wearable device may be removed, for example for charging thereof. In this regard, the same adhesive is used again for attaching the wearable device after being charged. Moreover, the medical grade adhesive is suitable for use by elderly patients whose skin is more fragile and sensitive with age, and prevents potential irritation associated with an extended exposure of the user's skin to the adhesive. Optionally, the adhesive is at least one of waterproof, water-resistant, sweatproof, sweat-resistant, and breathable in order to maintain comfort and cleanliness for the user. Beneficially, the waterproof and sweatproof adhesive enables use of the wearable devices in high moisture conditions such as, for example, during bathing, water activities and so forth, as well as during patient care for patients who are incapacitated such as wet wipe-off thereof. Eventually when the adhesive has been used for the required number of days, for example 14 days or more, the adhesive can be replaced with a new layer of adhesive.

Optionally, a peelable protective layer is arranged on the layer of adhesive when the wearable device is not in use. More optionally, the peelable protective layer is configured to cover and protect the adhesive when the wearable device is not adhered to the user. The peelable protective layer is configured to be removable from and replaceable on the adhesive (for example, by peeling the peelable protective layer away from the adhesive). Optionally, the peelable protective layer comprises a tab enabling the user to easily take hold of the peelable protective layer to remove the peelable protective layer from the adhesive and/or replace the peelable protective layer on the adhesive. Optionally, the peelable protective layer may be, or comprise, an acrylic film. Beneficially, the peelable protective layer prevents the adhesive from drying out or being potentially contaminated by foreign matter (for example, moisture, dust or a microorganisms) when the wearable device is not in use.

Optionally, the wearable device further comprises an electric charging portion to supply an onboard battery which powers the wearable device. The electric charging portion is configured to charge a rechargeable power source (such as the onboard battery) of the wearable device. Optionally, the electric charging portion is configured to receive electric power (for example electric power) from an external electric power source by a wireless or wired connection, for example a wireless resonant-inductive connection. In an example, for wireless charging, the electric charging portion may be or comprise one or more coils or windings (such as Litz wire or copper wire), disposed in the recess of the dissipating portion. In another example, the wearable device is charged using a wired connector (such as, for example, using pin charging method, such as USB charging or any other appropriate method) from an external electric power source. Typically, a receiving groove of the charging slot and a charging head of the wired connector serve as lock and key. The charging head may have various shapes and sizes including, but not limited to, a cylindrical pin (for example, POGO PIN) of various sizes, a USB (for example, USB-A, B or C), or a universal AC-DC connector. Optionally, the external electric power source may provide electric power to the wireless charging transmitter that in turn charges the wearable device wirelessly.

Optionally, the wearable device further comprises a modulation means. The term “modulation means” as used herein refers to a device configured to modulate an intensity of the electrical stimulus produced by the stimulating element, based on a user input. The modulation means, based on the user input, instructs the stimulating element to provide an output for operating the wearable device. The term “modulation” as used herein refers to varying or controlling one or more parameters associated with the operations of the wearable device based on a user's requirement. Typically, the electrical stimulus provided by the at least one stimulating element is modulated to vary an intensity thereof as per the user's requirements. In particular, such modulation of the intensity of the electrical stimulus changes one or more of a frequency, amplitude and phase of the electrical stimulus in proportion to an input received from the user. Optionally, the modulation is affected by a modulation means, such as a knob, a set of buttons, a touch application, provided on the wearable device. Optionally, the modulation means is provided on the distal surface of the wearable device. Alternatively, the modulation means may be provided on a side of the wearable device, for example on the housing. It will be appreciated that the modulation means is accessible to the users for control thereof.

The user input comprises one or more commands to instruct the at least one stimulating element to provide different outputs. Optionally, the user input comprises a first command and a second command, wherein the first and second commands are mutually different. Notably, the first command causes the at least one stimulating element to provide a first output; and the second command causes the at least one stimulating element to provide a second output. Optionally, the first output is at least one of a different length, frequency, intensity and change in intensity of an electrical stimulus from the second output. For example, the first output is a periodic output provided for a pre-determined period of time (for example, substantially between one minute and five minutes, such as substantially two minutes), whilst the second output is a substantially similar periodic output provided continuously for a longer duration (for example, substantially ten minutes). It will be appreciated that the different outputs may lead to different operations (i.e. for aiding specific tasks) of the wearable device, for example, in the above example the first output is directed at managing a severe tremor whilst the second output is directed at relaxing the muscle after the tremor has passed.

Optionally, the intensity of the electrical stimulus is selected from a continuous range of values between a maximum intensity and a medium intensity. The maximum intensity electrical stimulation fully controls the tremor, the medium intensity electrical stimulation relaxes the muscle experiencing tremors, and the intensities between the maximum and minimum intensities, such as a mid-level or low intensity electrical stimulation, enables controlling tremors in a subtle way. It will be appreciated that when the tremor is regulated, the device automatically stops sending further electronic stimulations, for example the maximum intensity and/or minimum intensity electrical stimulation, and keeps balancing the muscles by generating low intensity electrical stimulation to control a potential tremor. Therefore, the variation in intensity of the electrical stimulus produced by the stimulating element supports the wearable device in regulating stimulations applied to the individual based on the progress of the tremors.

Optionally, the wearable device further comprises a controller. The term “controller” herein refers to an electronic device. The electronic controller uses electrical signals and digital algorithms to perform its functions. The controller in the wearable device is configured to control an output of the at least one stimulating element. Optionally, the controller is coupled to one or more components of the wearable device. More optionally, the controller is coupled to the sensor. The controller receives sensor data and controls the output of the at least one stimulating element based on the sensor data. The controller manages the tremors by configuring at least two different sensor data, to instruct the stimulating element to provide at least two different outputs. Optionally, the controller is configured to receive at least a first sensor data and a second sensor data, wherein the second sensor data is different from the first sensor data. Notably, the first sensor enables the controller to instruct the at least one stimulating element to provide a first output and the second sensor data enables the controller to instruct the at least one stimulating element to provide a second output. In an example, the first sensor data and the second data may be a start of a tremor and an end of the tremor, respectively. Optionally, the second output is or comprises at least one of a different length, frequency, intensity and change in intensity of the electrical stimulus from the first output. The controller may support the stimulating element to produce different outputs and help regulate stimulations applied to the individual based on the progress of the tremors.

The present disclosure also relates to the kit of parts as described above. Various embodiments and variants disclosed above apply mutatis mutandis to the kit of parts.

The docking station, for receiving and storing the wearable device, is a single unit comprising a base and a lid that is removably attached at an end of the base. The term “docking station” as used herein refers to a storage unit of the wearable device, for example used to accommodate the wearable device when it is not being worn by a user. Optionally, the docking station may be used as an electrical charging unit for wireless or wired charging. Optionally, the docking station comprises a first portion (a base) and a second portion (a lid or closure). The first portion and the second portion may be releasably attachable, for example using corresponding male and female engagement features such as a fasteners (such as clamp fasteners), press-fit or interference fit, elastic clips and flanges, or complementary screw threads. In an embodiment, the first portion and the second potion are releasably attachable forming a single unit (or single body) in both closed and open conformations. In an alternate embodiment, the first portion and the second potion are two separate units that may be coupled together to form a single unit in a closed conformation. The first portion and the second portion may be connected together to form an internal recess to securely enclose the wearable device. The recess may be disposed in an upper surface of the first portion. A lower surface of the first portion may be substantially opposite the upper surface of the first portion and is used as a base for the docking station. Optionally, the recess comprises a receiving surface and a surrounding wall for receiving and preventing movement of the wearable device therefrom. Optionally, the receiving surface is inclined towards the surrounding wall at an acute angle (less than 90°, for example between substantially 15° and) 25° and the surrounding wall differs in height relative to the receiving surface around a perimeter of the receiving surface. Beneficially, the case may protect the wearable device during storage or transit.

Optionally, the kit of parts further comprises a mechanical engagement means that is configured to attach the wearable device on a given body part of the user. The mechanical engagement means is configured to enable attachment, detachment and/or reattachment of the wearable device to (or from) the body part of the user, i.e. a skin or muscle

Optionally, the docking station comprises a reservoir for adhesive. The term “reservoir” as used herein refers to a container, arranged within the docking station. The reservoir contains adhesive material for application thereof to the wearable device for attachment of the wearable device to skin of a user. Optionally, the reservoir is disposed in the first portion or the second portion of the docking station, and is rotatably removable from the docking station. Optionally, an outer surface (for example, an end wall or a side wall) of the docking station may be removable from the docking station to provide access to the reservoir (for example, for the user). Optionally, the outer surface comprises a notch enabling force to be applied to the part of the outer surface in order to remove it from the first portion. It will be appreciated that the size of the outer surface may be large enough to allow the wearable device (for example, a surface configured to deliver or transmit a mechanical stimulus to the user) to pass into the reservoir and contact the adhesive in the reservoir. This may enable easy application of additional adhesive to the wearable device as and when required. Optionally, the reservoir is refillable with adhesive material.

Optionally, the adhesive is provided as consumable pads. The consumable pads are typically distinct, separate layers of adhesive (for example, a stack of adhesive layers) which are individually retrievable from the reservoir and which may be applied separately to the wearable device. Optionally, a predefined number of consumable pads, for binding to the wearable device, may be provided in the reservoir. The unique design of the reservoir enables accessing the stored consumable pads of adhesive when the consumable pads of adhesive presently in use with the wearable device is unable to show the desired effect. The rotating outwards of the reservoir enables keeping the consumable pads of adhesive in place and enhancing the longevity of the docking station by subjecting it to least amount of jerks and orientational changes.

Optionally, the consumable pads are fabricated from a hook layer and a loop layer, such as a Velcro™, comprising layers of adhesives (of different grades and strength) on the back side thereof. A medical grade adhesive is provided at the back of the loop layer that is placed facing towards the skin of the user, and a stronger adhesive (than the medical grade adhesive) is provided at the back of the hook layer that is placed facing towards the wearable device, such as on the dissipating portion thereof, for binding with the wearable device. The hook layer and the loop layer are configured to couple to each other by means of tiny hooks in the hook layer and smaller loops in the loop layer, such that the hooks catch in the loops and the two layers fasten or bind temporarily. Optionally, the hook and loop layers are separated by pulling or peeling apart the loop layer from the hook layer.

The wearable device is placed onto the skin of the user using the consumable adhesive pad. The wearable device can be removed for charging or when not required for use, with the loop layer of the consumable adhesive pad remaining attached on the skin via the medical grade adhesive, while hook layer of the consumable adhesive pad remains attached on the wearable device via the stronger adhesive. This allows using the same consumable adhesive pad, for a predefined time, while the wearable device is being charged. Eventually when the consumable adhesive pad has been used for the required number of days, for example 14 days or more, such as 20 days, the consumable adhesive pad can be removed and a new consumable adhesive pad can be applied using the process described above. Alternatively, the consumable adhesive pad may be replaced if the consumable adhesive pad fails to provide desired adhesion on the skin of the user or the wearable device. Moreover, the hook layer is strong enough to last several months if not years. However, the hook layer can also be detached from the wearable device in case the hook layer wears off.

The present disclosure also provides a method of operating a wearable device for managing tremors emanating from a body part of a user, wherein the method comprises:
configuring a sensor of the wearable device to detect a tremor and transmit corresponding sensor data to at least one stimulating element of the wearable device;
configuring the at least one stimulating element to provide an electrical stimulus based on the sensor data; and
configuring a dissipating portion of the wearable device to increase an effective area for dissipating the electrical stimulus to the body part, wherein the dissipating portion is physically coupled with the at least one stimulating element,
   wherein the method further includes positioning the wearable device, when in operation, in physical contact with the body part of the user wherefrom the tremors emanate.

Optionally, the method comprises arranging the wearable device in physical contact with the body part of the user wherefrom the tremors emanate. In this regard, the user may arrange the wearable device at the target body part themselves or with the help of a user's carer. The wearable device, when in operation, detects a tremor using the sensor thereof and transmit corresponding sensor data to at least one stimulating element. The at least one stimulating element provides an electrical stimulus based on the sensor data and the dissipating portion dissipates the electrical stimulus to the body part.

The present disclosure also relates to the device integration application as described above. Various embodiments and variants disclosed above apply mutatis mutandis to the device integration application.

The device integration application comprises a software application that is executed by a data processing arrangement of a device. Optionally, the wearable device uses a software application that is integrated with an external device (referred to as “device” hereafter). Such to-use application software integrated in at least one compatible device is referred to as the device integration application. The term “device integration application” refers to a software program for execution by the device by employing the processor of the device. Notably, the device integration application comprises a set of predefined functions that are programmed to provide instructions to hardware and/or software elements of the device. Furthermore, the device integration application is configured to provide a user interface on the display of the device, to allow the user to perform specific associated tasks. In an example, the device integration application is an application programming interface. In an embodiment, the device integration application is affiliated to an organization, for example a hospital or health provision service. Therefore, the device integration application functions in accordance with pre-programmed guidelines provided by the organization. The device integration application is configured to function in accordance with the pre-programmed guidelines upon installation thereof. The device integration application typically enables spatial tracking of the wearable device, when in operation. The at least one compatible device that is configured to run the device integration application thereon includes, but is not limited to, a mobile, a laptop computer, a tablet computer, a desktop computer, a palmtop computer and a smart watch.

Optionally, the device integration application comprises a set of activities for measuring the quality of life of the user. Optionally, the set of activities include games, assessment, commands, reminders, and so on for monitoring the progression of symptoms, such as tremors, associated with the disease. More optionally, the set of activities monitor dexterity and focus of a patient and information generated therefrom may be used by a third-party, such as a healthcare professional, a family or a researcher, to monitor a progression of symptoms in the patient. Optionally, the device integration application is configured to collect and analyse the data recorded by the user and generates any of: graphs, reports, day-to-day analysis, and so on to be shared with the user's doctor or physician via a communication network, such as internet, Bluetooth® connection, servers, and so on.

In an example, the device integration application includes activities to help the users to fight psychological isolation. Activities, such as drawing a spiral and/or a straight line between the two points using their fingers or a stylus pen or identifying and tracking a change in the display of the screen, are designed to test speed, accuracy and dexterity of the user. The user can take multiple attempts that helps in improving movement. In another example, a self-assessment questionnaire enables the user to fill out answers to the questions therein routinely, for example weekly, to track and inform a doctor or carer about the user's health. The questionnaire may require the user to choose from a predefined scale of responses to register the progression of their symptoms. In yet another example, the device integration application enables the user to instruct the device to remind the user or the user's carer to ensure that the user takes prescribed medicines, for example, if the details, such as names of medicines, doses of medicines, time of taking medicine, and so on, are furnished therein. Optionally, the reminder may comprise an alert pattern, such as visual, audible, or a combination thereof, for the family or carer of the user to help an incapacitated user. Moreover, the user may register acknowledgement for the aforesaid reminder using the device integration application. Additionally, the device integration application allows scheduling appointments with the doctor and sending an advanced notification (such as 2 days, 1 day, 6 hours, and 2 hours before the due appointment) therefor.

Optionally, the device integration application is executable to implement the aforementioned method. Optionally, the device integration application allows the user to modulate the intensities of the electrical stimulus. Optionally, the device integration application enables the user to apply touch or light pressure for accessing the wearable device when in operation. The touch or light pressure application by the user on the user input is configured to provide instructions to any of the controller or modulation means to instruct the at least one stimulating element to provide an output based on the received user input. The user input is directed at selecting a range of intensities of electrical stimulation to manage tremors.

### DETAILED DESCRIPTION OF THE DRAWINGS

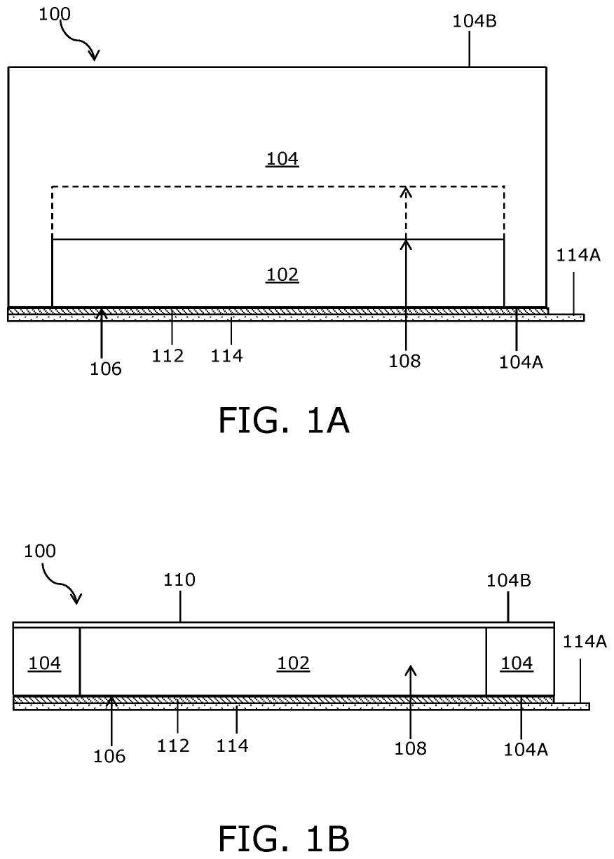

Referring to FIGS. 1A and 1B, there are provided schematic illustrations of a wearable device 100, in accordance with various embodiments of the present disclosure. The wearable device 100 for managing tremors emanating from a body part of a user comprises a sensor (not shown) that is configured to detect a tremor and transmit sensor data to at least one stimulating element, such as the stimulating element 102. The at least one stimulating element, such as the stimulating element 102, is configured to provide an electrical stimulus based on the sensor data. The wearable device 100 also comprises a dissipating portion 104 configured to increase an effective area for dissipating the electrical stimulus to the body part. The dissipating portion 104 is physically coupled with the at least one stimulating element, such as the stimulating element 102. The at least one stimulating element, such as the stimulating element 102, may be provided or housed in the dissipating portion 104. The dissipating portion 104 has a proximal end 104A and a distal end 104B. The proximal end 104A of the dissipating portion 104 forms at least a part of a proximal surface 106 of the wearable device 100 that is configured to contact skin of the user and to transmit the mechanical stimulus from the at least one stimulating element, such as the stimulating element 102, to the user. The distal end 104A of the dissipating portion 104 comprises a recess 108 (as indicated by the dashed lines in FIG. 1A). The at least one stimulating element, such as the stimulating element 102, is configured to be received within the recess 108. As shown, the at least one stimulating element, such as the stimulating element 102, also forms at least a part of the proximal surface 106. In this way, the dissipating portion 104 is configured to surround the at least one stimulating element, such as the stimulating element 102, except for at the distal end 104A, where the at least one stimulating element, such as the stimulating element 102, is left exposed. The distal end 104B of the dissipating portion 104 comprises a closed end of the dissipating portion 104 and forms an outer or distal surface of the wearable device 100. The proximal surface 106 comprises, or is provided with, a layer of adhesive 112 that is configured to adhere the proximal surface 106 to the skin of the user. A peelable protective layer 114 is arranged on the layer of adhesive 112 when the wearable device 100 is not in use. The peelable protective layer 114 comprises a tab 114A for enabling the user to easily take hold of the peelable protective layer 114 to remove the peelable protective layer 114 from the adhesive 112 and/or to replace the peelable protective layer 114 on the adhesive 112 when the wearable device 100 is not in use.

In an alternative embodiment, as shown in FIG. 1B, the distal end 104B of the dissipating portion 104 is open, and the recess 108 extends through the dissipating portion 104 to the open second end 104B to form an aperture (e.g., a recess open at both ends) through the dissipating portion 104. In such an embodiment, an additional capping layer 110 is provided to ensure the at least one stimulating element, such as the stimulating element 102, and any other components of the wearable device 100 remains secured or located within the recess or aperture 108.

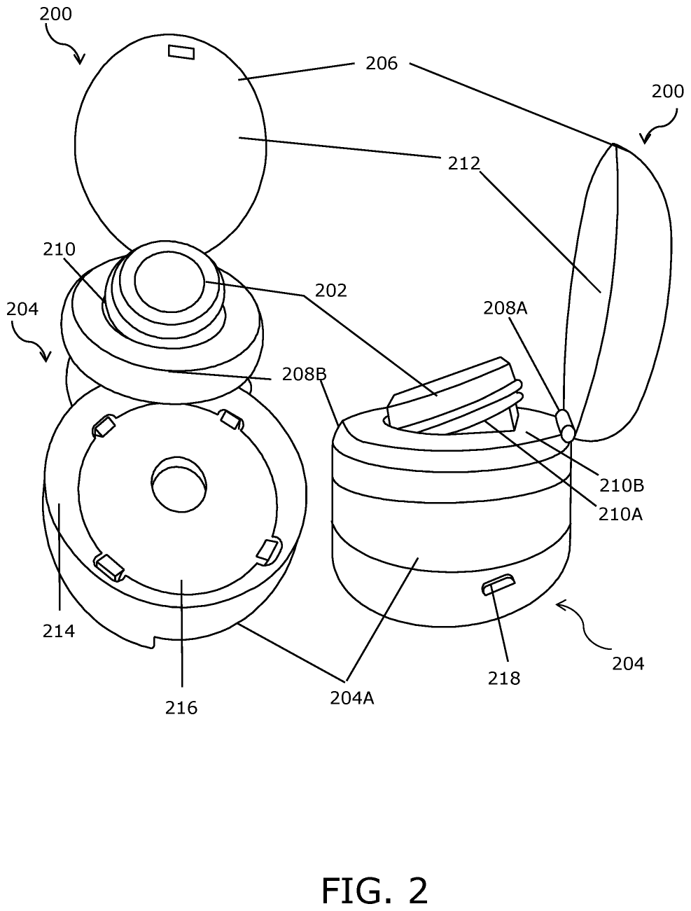

Referring next to FIG. 2, there is provided a schematic illustration of a docking station 200, in accordance with an embodiment of the present disclosure. The docking station 200 is configured to hold and retain the wearable device 202 when the wearable device 202 is not being worn by the user. As shown, the docking station 200 is a single unit comprising a first portion (or base) 204 and a second portion (or lid or cover) 206 that is attached to the first portion 204. The first portion 204 and the second portion 206 are releasably attachable to one another at a first end 208A and close at a second end 208B (using pieces of magnet at the closing ends for example) opposite the first end 208A. The first portion 204 comprises an outer surface 204A and an inner surface (not shown).

The first portion 204 of the docking station 200 comprises a recess 210 that is configured to receive the wearable device 202. The recess 210 comprises a receiving surface 210A and a surrounding wall 210B for receiving and preventing the movement of the wearable device 202 therein. The receiving surface 210A is inclined towards the surrounding wall 210B at an acute angle and the surrounding wall 210B differs in height relative to the receiving surface 210A around a perimeter (on one half of the perimeter) of the receiving surface 210A. The second portion 206 comprises a recess 212 which is complementary to the recess 210 of the first portion 204. Together, when the second portion 206 is attached to the first portion 204, at the second end 208B, the recess 212 and the recess 210 form an internal recess of the docking station 200 which is configured to securely enclose the wearable device 202 within the internal.

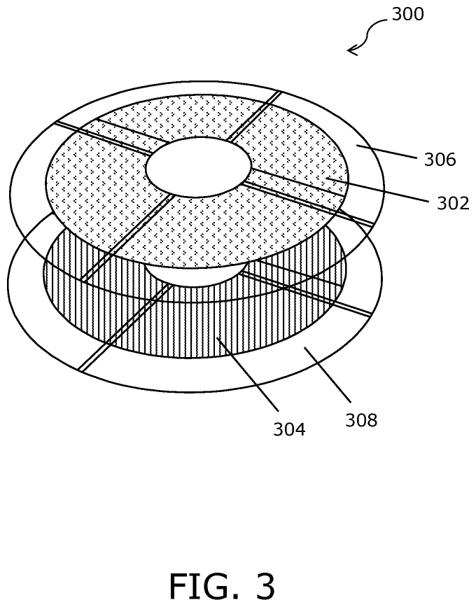

Furthermore, the first portion 204 of the docking station 200 comprises a reservoir 214 that is configured to contain additional adhesive. The reservoir 214 is provided in a part of the outer surface 204A of the first portion 204. The part of the outer surface 204A of the first portion 204 is replaceably removable from the first portion 204 using a notch (not shown) enabling force to be applied to the part of the outer surface 204A in order to remove it from the first portion 204 to provide access to the reservoir 214. The size of the reservoir 214 is complimentary to the size of the wearable device 202 to allow easy access to the adhesive 216 therein. The additional adhesive may be provided to the reservoir 214 in distinct, separate layers of adhesive (such as consumable adhesive pads as shown in FIG. 3) which are individually retrievable from the reservoir 214 and which may be applied separately to the wearable device 202. In some embodiments, the reservoir 214 is refillable.

The docking station 200 comprises an electric power connection 218 that is disposed on the first portion 204 and is configured to connect the docking station 200 to an external electric power source (for example, to a mains electricity supply).

Referring to FIG. 3, there is provided a schematic illustration of a consumable adhesive pad 300, in accordance with an embodiment of the present disclosure. The consumable adhesive pad 300 comprises a hook-and-loop fastener, such as a Velcro™, possessing adhesive. The hook-and-loop fasteners comprise a hook layer 302 and a loop layer 304. The hook layer 302 and the loop layer 304 are configured to couple to each other by means of tiny hooks in the hook layer 302 and smaller loops in the loop layer 304. When the hook layer 302 and the loop layer 304 are pressed together, the hooks catch in the loops and the two layers fasten or bind temporarily. The two layers are separated by pulling or peeling apart the loop layer 304 from the hook layer 302. The respective backs of the hook layer 302 and the loop layer 304 comprise adhesives, namely a first layer of adhesive 306 and a second layer of adhesive 308, respectively. The consumable adhesive pad 300 is arranged to attach to the skin of the user by the second layer of adhesive 308 that is coupled to the loop layer 304 and to the surface of the wearable device (such as the wearable device 100 of FIG. 1 or 200 of FIG. 2) by the first layer of adhesive 306 that is coupled to the hook layer 302. The adhesives 306 and 308 on the hook layer 302 and the loop layer 304 respectively are of mutually different grades and strength. As such, the second layer of adhesive 308 is a medical grade adhesive for binding with the skin of the user, and the first layer of adhesive 306 is a stronger adhesive (than the medical grade adhesive) for binding with the wearable device. The consumable adhesive pad 300 is arranged in the reservoir (such as the reservoir 214 of FIG. 2) such that the side corresponding (or attachable) to the surface of the wearable device is exposed from the reservoir. When the wearable device 202 is pressed in the reservoir, a consumable adhesive pad 300 is transferred on to the surface of the wearable device. The consumable adhesive pad 300 comprises a peelable protective layer (such as the peelable protective layer 114 of FIG. 1) with a tab to enable easy removal of the peelable protective layer.

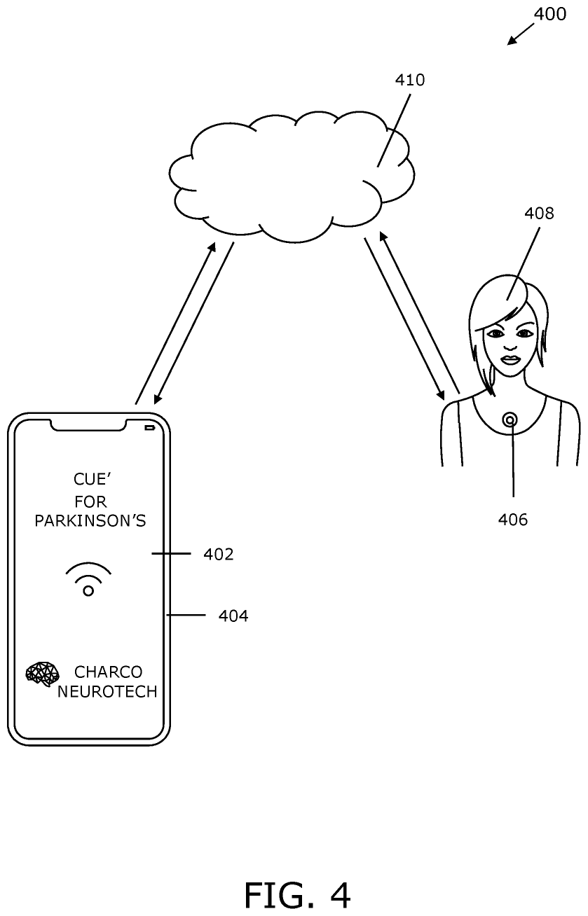

Referring to FIG. 4, there is provided an exemplary implementation 400 of a successful installation of the device integration application 402 on a device 404, such as a smart phone. As shown in FIG. 4, the device integration application 402 enables controlling operations of a wearable device 406 when in use by a user 408 and/or receive the information from the wearable device 406 and/or the user 408 to be used to monitor progress of a disease in the user 408. The device 404 and the wearable device 406 are communicably coupled via a communication network 410. The device integration application 402 enables sending the data recorded by the user 408 to the third party, such as a physician, a carer, or a server, for tracking the progress of the user 408.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A wearable device for managing tremors emanating from a body part of a user, the wearable device comprising:
a sensor that is configured to detect a tremor and transmit corresponding sensor data to at least one stimulating element;
the at least one stimulating element that is configured to provide an electrical stimulus based on the sensor data; and
a dissipating portion that is configured to increase an effective area for dissipating the electrical stimulus to the body part, wherein the dissipating portion is physically coupled with the at least one stimulating element,
wherein the wearable device, when in operation, is in physical contact with the body part of the user wherefrom the tremors emanate.

2. The wearable device of claim 1, wherein the sensor data is at least one of: a start of tremor, an end of tremor, a severity of tremor, a frequency of tremor, locus of tremor.

3. The wearable device of claim 1 or 2, wherein the wearable device is configured to regulate, based on the sensor data, the at least one stimulating element to control one or more parameters of the electrical stimulus.

4. The wearable device of claim 1 any one of the preceding claims, further comprising a modulation means configured to modulate an intensity of the electrical stimulus based on a user input.

5. The wearable device of claim 4, wherein the intensity of the electrical stimulus is selected from a continuous range of values between a maximum intensity and a minimum intensity.

6. The wearable device of claim 1, wherein the physical contact with the body part of the user is by a mechanical engagement means, and wherein the mechanical engagement means is at least one of: an adhesive, a strap, a locket, a bracelet, a band, a belt, a vacuum cup, a magnet, a hook and loop fastener.

7. The wearable device of claim 1, wherein the body part is a skin of body parts selected from at least one of: a head, a sternum, an arm, a shoulder, a wrist, a hand, a neck, a jaw, a trunk, an ankle, a leg, foot and a temple.

8. The wearable device of claim 1 any one of the preceding claims, further comprising an electric charging portion to supply an onboard battery which powers the wearable device.

9. The wearable device of claim 8, wherein the electric charging portion is configured to receive electric power from an external electric power source by a wired connection or wirelessly.

10. The wearable device of claim 1, wherein the tremor is associated with at least one condition selected from: Parkinson's disease, Alzheimer's disease, essential tremor, multiple sclerosis, neural infection, neural death or damage, a shock, a stroke or neurodegenerative disease.

11. The wearable device of claim 1, wherein the sensor is selected from at least one of: a gyroscopic sensor, an accelerometer a sensor, a magnetometer sensor, an electromyography sensor, a flexural sensor, a stretch sensor, or a combination thereof.

12. A kit of parts comprising:
a wearable device of claim 1; and
a docking station for receiving and storing the wearable device, wherein the docking station is a single unit comprising a base and a lid removably attached at an end of the base.

13. A kit of parts of claim 12, further comprising a mechanical engagement means configured to attach the wearable device on a given body part of the user.

14. A method of operating a wearable device for managing tremors emanating from a body part of a user, wherein the method comprises:
configuring a sensor of the wearable device to detect a tremor and transmit corresponding sensor data to at least one stimulating element of the wearable device;
configuring the at least one stimulating element to provide an electrical stimulus based on the sensor data; and
configuring a dissipating portion of the wearable device to increase an effective area for dissipating the electrical stimulus to the body part, wherein the dissipating portion is physically coupled with the at least one stimulating element,
wherein the method further includes position the wearable device, when in operation, in physical contact with the body part of the user wherefrom the tremors emanate.

15. A device integration application comprising a software application to be executed by a data processing arrangement of a device, wherein the device is communicably coupled to a wearable device of claim 1.

16. The device integration application of claim 15, wherein the device integration application allows the user to modulate one or more parameters of electrical stimulus provided by the wearable device.

17. The device integration application of claim 15, wherein the device integration application comprises a set of activities for measuring the quality of life of the user.

18. The device integration application of claim 15, wherein the device integration application is executable to implement the method of claim 14.
